(19) United States
(12) Patent Application Publication
(10) Pub. No.: US 2018/0235544 A1
(43) Pub. Date: Aug. 23, 2018

(54) IMPLANTABLE SENSOR ASSEMBLY SYSTEMS AND METHODS

(71) Applicant: General Electric Company, Schenectady, NY (US)

(72) Inventors: Kaustubh Ravindra Nagarkar, Clifton Park, NY (US); Jeffrey Michael Ashe, Gloversville, NY (US); Eric Patrick Davis, Wynantskill, NY (US); Nancy Cecelia Stoffel, Schenectady, NY (US)

(21) Appl. No.: 15/625,740

(22) Filed: Jun. 16, 2017

Related U.S. Application Data
(60) Provisional application No. 62/460,129, filed on Feb. 17, 2017.

(51) Int. Cl. A61B 5/00; H05K 5/00; H05K 1/18; H05K 1/11; H05K 5/02; H05K 3/30; H05K 3/40; A61B 5/07; C23C 14/34; B33Y 80/00

(52) U.S. Cl. CPC A61B 5/6861; H05K 5/0026; H05K 1/181; H05K 1/113; H05K 5/0247; H05K 1/0313; H05K 5/0095; H05K 3/4038; A61B 5/076; C23C 14/34; B33Y 80/00; H05K 3/303

(57) ABSTRACT

A system includes an implantable sensor assembly. The implantable sensor assembly includes a housing. The housing includes a substrate layer comprising an interior surface and an exterior surface, and a cap layer, wherein the substrate layer and the cap layer are coupled to form an enclosed cavity that at least partially encloses the interior surface of the substrate layer within the cavity and wherein both the substrate layer and the cap layer are formed from an insulating material. The implantable sensor assembly also includes one or more electronic components disposed within the cavity of the housing and one or more probes disposed on the exterior surface of the substrate layer and electrically coupled to the one or more electronic components by one or more electrical connections extending through the housing.

### CROSS-REFERENCE TO RELATED APPLICATION

This application claims priority to and the benefit of U.S. Provisional Application No. 62/460,129, entitled “ENCAPSULATED ELECTRONICS FOR NEURAL AND OTHER MEDICAL IMPLANTS”, filed Feb. 17, 2017, which is herein incorporated by reference in its entirety for all purposes.

### BACKGROUND

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

A variety medical diagnostic, treatment, and therapeutic processes utilize implant structures (e.g., microelectrode array assemblies) to record, process, and transmit neural signals from a subject's brain or other sources to an external data acquisition system for interpretation. The commercially available implants typically include electrical sensors and are directly wired through the skull to signal processing and communication electronics. To be implantable into the brain, or other location within the body, the sensing implant structures are packaged into materials appropriate for implantation. Typical packaging materials used include titanium, a biocompatible and strong, although rigid and thick material, and polymeric materials, such as epoxies and silicones, which are flexible, although porous and not entirely hermetic. Sensing implant structures do not have high survival rates for chronic implantation. The failure reasons include the failure of the electronic assemblies of the sensing implant structures in the body. Additionally, some sensing structures (e.g., probes) are first individually manufactured and then assembled into an array. This process is slow, causes yield loss, and is not amenable to integration with processing electronics.

### BRIEF DESCRIPTION

Certain embodiments commensurate in scope with the originally claimed subject matter are summarized below. These embodiments are not intended to limit the scope of the claimed subject matter, but rather these embodiments are intended only to provide a brief summary of possible embodiments. Indeed, the disclosure may encompass a variety of forms that may be similar to or different from the embodiments set forth below.

In one embodiment, a system includes an implantable sensor assembly. The implantable sensor assembly includes a housing. The housing includes a substrate layer comprising an interior surface and an exterior surface, and a cap layer, wherein the substrate layer and the cap layer are coupled to form an enclosed cavity that at least partially encloses the interior surface of the substrate layer within the cavity and wherein both the substrate layer and the cap layer are formed from an electrically insulating material. The implantable sensor assembly also includes one or more electronic components disposed within the cavity of the housing and one or more probes disposed on the exterior surface of the substrate layer and electrically coupled to the one or more electronic components by one or more electrical connections extending through the housing.

In a second embodiment, a system includes an implantable sensor assembly. The implantable sensor assembly includes a substrate layer, a cap layer comprising a recess on one side, a cavity formed between the substrate layer and the cap layer, wherein the recess of the cap layer forms part of the cavity, a seal disposed between the substrate layer and the cap layer, wherein the seal is configured to seal the cavity, wherein the seal, cap layer, and substrate layer are formed from electrically insulating materials, one or more electronic components disposed on a substrate platform, wherein the substrate platform is disposed on an interior surface of the substrate layer within the cavity, and one or more probes disposed on an exterior surface of the substrate layer such that the probes are outside of the cavity.

In a third embodiment, a method for fabricating an implantable sensor assembly includes providing a substrate layer, wherein the substrate layer comprises one of glass or liquid crystal polymer (LCP), attaching electronic components directly to a first surface of the substrate layer, sealing a cap layer over the first surface of the substrate layer to create a cavity between the substrate layer and the cap layer, wherein the cap layer comprises one of glass or liquid crystal polymer (LCP), and coupling one or more probes to a second surface of the substrate layer opposing the first surface and such that the one or more probes are disposed outside of the cavity.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 depicts a process flow of fabrication of an embodiment of an implantable sensor assembly having through via connections, in accordance with aspects of the present disclosure;

FIG. 2 depicts a process flow of fabrication of an embodiment of an implantable sensor assembly having a side feed-through connection, in accordance with aspects of the present disclosure;

FIG. 3 depicts a process flow of fabrication of an embodiment of an implantable sensor assembly incorporating a sensor module built using traditional processes, in accordance with aspects of the present disclosure;

FIG. 4 depicts a cross section view of an embodiment of an implantable sensor assembly having a side feed-through connection, in accordance with aspects of the present disclosure;

FIG. 5 depicts a cross section view of an embodiment of an implantable sensor assembly having no feed-through connections, in accordance with aspects of the present disclosure;

FIG. 6 depicts a cross section view of an embodiment of an implantable sensor assembly having through via connections, in accordance with aspects of the present disclosure;

FIG. 7 depicts a cross section view of an embodiment of an implantable sensor assembly incorporating a sensor module, in accordance with aspects of the present disclosure;

FIG. 8 depicts a cross section view of an embodiment of an implantable sensor assembly having multiple through via connections, in accordance with aspects of the present disclosure;

FIG. 9 depicts a cross section view of an embodiment of an implantable sensor assembly having an attached probe array, in accordance with aspects of the present disclosure;

FIG. 10 depicts a cross section view of an embodiment of an implantable sensor assembly having directly connected probes, in accordance with aspects of the present disclosure;

FIG. 11A depicts a bottom view of an embodiment of an implantable sensor assembly, in accordance with aspects of the present disclosure;

FIG. 11B depicts a bottom view of an embodiment of an implantable sensor assembly, in accordance with aspects of the present disclosure; and

FIG. 12 depicts a schematic diagram of an embodiment of a control system that may be employed with an implantable sensor assembly, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Provided herein are sensing implant structures that may be used to record, process, and transmit neural signals from the brain or other sources to an external data acquisition system for interpretation during medical diagnosis and/or treatment. The disclosed implant structures may also be used to provide treatment, e.g., may be used for neurostimulation. The sensing implant structures may be packaged or encapsulated in biocompatible materials to enclose any incorporated electronic components. While certain embodiments of the disclosure are disclosed in the context of probes or neural probes, it should be understood that the disclosed techniques may be incorporated into other implantable medical devices, such as pacemakers, pumps, and other sensing and actuation devices or stimulation devices. Certain disclosed embodiments are directed toward an implantable sensor assembly. The implantable sensor assembly may include a first substrate layer or layers that may serve as a circuit board for an electronics assembly and/or an interposer between encapsulated electronics and a probe array and that also may serve as an environmental protection layer (e.g., hermetic layer) for the enclosed electronic components. By forming or positioning the electronic components of the assembly directly on the environmental protection layer rather than on a separate circuit board or electronics layer, the overall profile of the device is reduced. The implantable sensor assembly may include a second cap layer use to encapsulate the electronic components within a cavity formed by the first substrate layer and the second cap layer. The first substrate layer and the second cap layer may be made from biocompatible materials, including glass, liquid crystal polymer (LCP), fused silica, quartz, sapphire, or any other suitable material. These materials may enable a thinner and lower profile implantable sensor assembly relative to assemblies that use additional layers, e.g., formed from thicker and more rigid metal materials. Further, the implantable sensor assembly may include through connections, via side feed-through connections and/or through via connections, for electrical connection of the enclosed electronics with coupled probe assemblies and/or other electronics.

Further, the disclosed embodiments provide methods for fabricating the implantable sensor assembly. In one embodiment, the implantable sensor assembly may be fabricated with through via connections. In one embodiment, the implantable sensor assembly may be fabricated with side feed-through connections. In one embodiment, the implantable sensor assembly may be fabricated with sensor modules built using traditional processes and positioned within the cavity formed by environmental protection layers of the implantable sensor assembly. These methods may enable increased modularity and improved manufacturability of the implantable sensor assembly, thus facilitating a reduction in cost of the implantable sensor assembly.

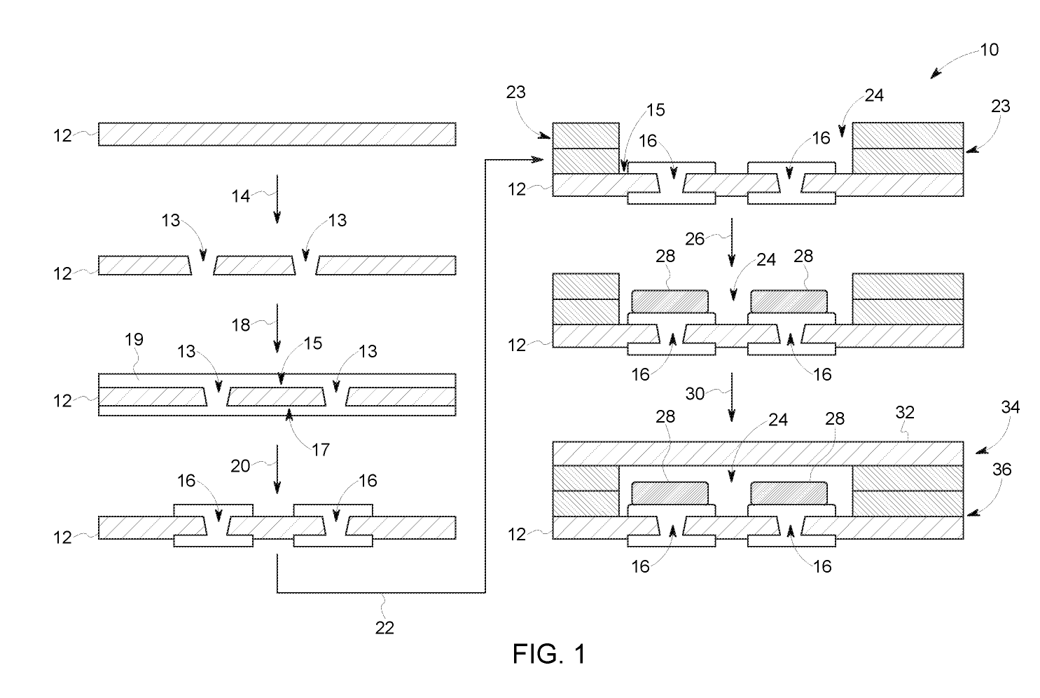

With the preceding in mind, FIGS. 1-3 depict three process views showing fabrication of implantable sensor assemblies having cavities for encapsulating electronic components of the implantable sensor assembly. To illustrate, FIG. 1 shows fabrication steps (process 10) for fabricating an embodiment of an implantable sensor assembly 34 having through via connections 16. At a first step, a substrate layer 12 is provided. The substrate layer 12 (e.g., platform layer) may serve as a portion of a biocompatible encapsulating housing 36 of the implantable sensor assembly 34. In certain embodiments, the substrate layer 12 may be made of such materials as glass or liquid crystal polymer (LCP), or any other suitable material. The biocompatible encapsulating housing 36, including the substrate layer 12, may be made of any material approved to be implanted in the body (e.g., does not react with the body), having a low permeability to moisture or ions (e.g., near hermetic or water impermeable), having insulator capability (e.g., insulating dielectric), and may enable the implantable sensor assembly to be less than approximately 1 millimeter tall. The insulating capability of the biocompatible encapsulating housing 36 may be described as an electrical resistivity (e.g., electrical resistance) of the substrate material. The electrical resistivity of the substrate material may indicate how strongly the material opposes a flow of electric current. The electrical resistivity of the substrate material may be measured in Ohm-meters (Ωm), with a higher value corresponding to a higher electrical resistivity. For example, glass as a substrate material may have a relatively high resistivity value of approximately 10×10¹⁰ to 10×10¹⁴ Ωm, thus glass may have a high insulating capability. In the illustrated embodiment, the substrate layer 12 is a single layer; however, the substrate layer may be any quantity (e.g., 2, 3, 4, or more) of laminated layers of the substrate material. In a next step 14, the substrate layer 12 may be drilled, cut, machined, or otherwise formed to create passageways 13 (i.e., through vias) through the substrate layer 12 of the implantable sensor assembly 34. The passageways accommodate through via connections 16 that may be used to electrically connect any encapsulated electronics with a coupled probe array and/or other electronics. In the illustrated example, two passageways 13 have been formed, however, any quantity (e.g. 3, 4, 5, 6, or more) may be formed into the substrate layer 12, and thus be present in the implantable sensor assembly 34.

At a next step 18, the substrate layer 12, having passageways 13 for through via connections 16, may be metallized with a metal layer 19 on an interior surface 15 and an exterior surface 17 of the substrate layer 12. In a step 20, the metal layer 19 may be patterned to surround and fill the passageways 13 to form the through via connections 16. Metallization of and around the passageways 13 by the through via connections 16 may enable the implantable sensor assembly 34 to be completely or substantially sealed while enabling connection to a coupled probe array and/or outside electronics outside of the implantable sensor assembly 34. The through via connections 16 extend through the substrate layer 12 from the top interior surface 15 to the opposing bottom exterior surface 17. In a next step 22, in some embodiments, a cavity 24 may be formed through lamination of additional layers of the biocompatible substrate material around the edges of the interior surface 15 of the substrate layer 12. This lamination step 22 may form sides 23 of the implantable sensor assembly 34 and, thus, sides 23 of the cavity 24. The sides 23 may act as a seal to seal the substrate layer 12 to other components, e.g., the seal layer 32. Traditional lamination techniques may be used build the sides 23, and, thus, the cavity 24, to an expected depth. In other embodiments, the cavity 24 may be formed through mechanical means, such as ultrasonic machining, sandblasting, chemical etching, embossing, additive manufacturing, or any other process suitable for forming the cavity 24 in the substrate layer 12. The cavity 24 may be formed by the substrate layer and the sides 23, and may be the housing space for electronic components 28 (e.g., signal processing and/or transmission electronics) of the implantable sensor assembly 34. In a next step 26, the electronic components 28 of the implantable sensor assembly 34 may be attached to the metal layer and/or directly to the substrate layer 12 inside the cavity 24. The electronic components 28 may be positioned inside the cavity 24 above or near the through via connections 26 such that connections to outside probes and/or electronics may be made. As previously mentioned, the substrate layer 12 may serve as a circuit board for the attached electronic components 28 of the implantable sensor assembly 34. Additionally, the patterned metal layer 19 may enable connection between the individual encapsulated electronic components 28. In the depicted step 30, the cavity 24 may be sealed by adding a cap layer 32. Like the substrate layer 12, the cap layer 32 may be made of glass or liquid crystal polymer (LCP), or any other material suitable for creating the biocompatible encapsulating housing 36. In certain embodiments, the cap layer 36 and the substrate layer 12 may be formed from the same or different materials. In some embodiments, the cap layer 32 may be a flat layer. In other embodiments, the cap layer 32 may contain a recess that becomes part of the cavity 24, such that the cap layer 32 also forms the sides 23. Thus the cap layer 32 may be a single layer including the sides 23, or the cap layer 32 and the sides 23 may be formed from different layers. In some embodiments, the cavity 24 may be formed through mechanical means, such as ultrasonic machining, sandblasting, chemical etching, embossing, additive manufacturing, or any other process suitable for forming the cavity 24 in the cap layer 32. The cap layer 32 may be added and the perimeter (e.g., substrate layer 12, sides 23, and cap layer 32) of the implantable sensor assembly 34 may be sealed using a localized perimeter sealing technique. The localized perimeter sealing technique may be a technique that will not damage the encapsulated electronic components 28. For example, in some embodiments, the localized perimeter sealing technique may be a low temperature technique (e.g., laser seal) that does not use harsh solvents. In some embodiments, the localized perimeter sealing technique may concentrate the heat used in the sealing process in only the seal region, such that the heat is away from the encapsulated electronic components 28.

The resulting implantable sensor assembly 34 contains the substrate layer 12, laminated sides 23, and the cap layer 32, each made of glass or liquid crystal polymer (LCP), or any other material suitable for creating the biocompatible encapsulating housing 36. The implantable sensor assembly 34 contains the cavity 24 within the biocompatible encapsulating housing 36. Within the cavity 24, the implantable sensor assembly 34 contains the electronic components 28 attached to the metal layer and/or the substrate layer 12. The implantable sensor assembly 34 includes the metalized through via connections 16 formed through the substrate layer 12 that may enable connection between the encapsulated electronic components 28 and probes that may be coupled to the implantable sensor assembly 34, other outside electronics, and/or other implantable sensor assemblies. The implantable sensor assembly 34 may enable increased reliability of the sensor electronic components 28 by encapsulating and protecting the electronic components 28 from the environment outside of the implantable sensor assembly 34 and permitting connection to other electronics through the through via connections 16. Further, the process 10 may enable increased modularity and improved manufacturability of the implantable sensor assembly 34.

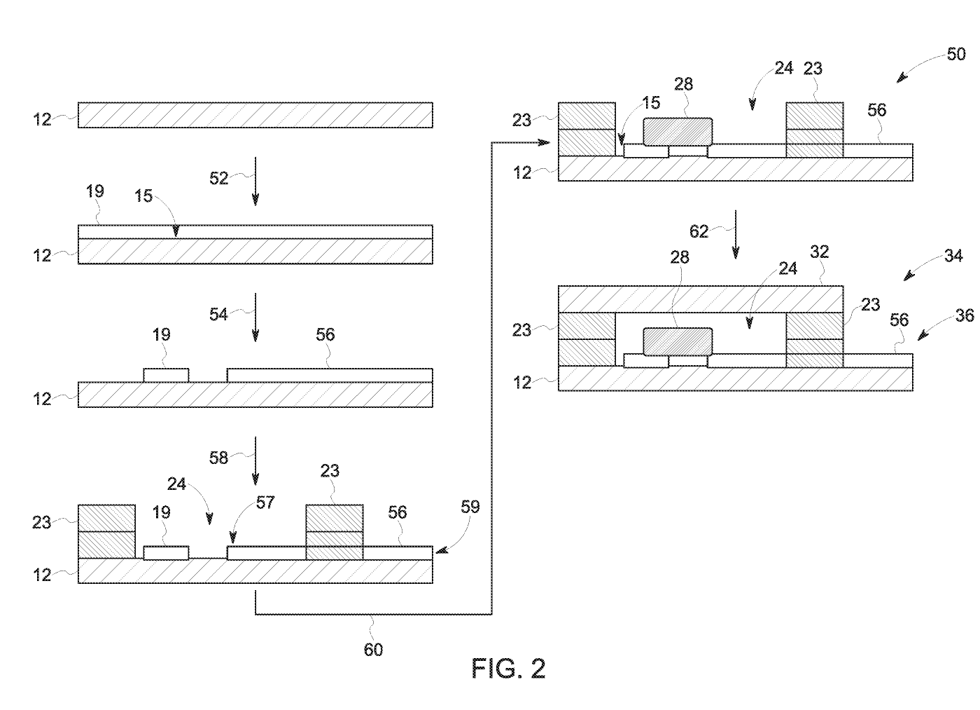

FIG. 2 depicts a process flow (process 50) suitable for fabricating an embodiment of the implantable sensor assembly 34 having side feed-through connections 56. Side feed-through connections 56 may enable a further electrical or sensor interface for connecting the implantable sensor assembly 34 with outside electronics and/or other implantable sensor assemblies. Such connections may enable increased modularity and integration of the implantable sensor assembly 34 with other sensor assemblies and/or processing electronics. FIG. 2 depicts the implantable sensor assembly 34 having only a side feed-through connection, and FIG. 1 depicts the implantable sensor assembly 34 having only the through via connections 16, however it should be understood that the implantable sensor assembly 34 may have any combination of the side feed-through connections 56 and the through via connections 16.

At a first step, the substrate layer 12 is provided. The substrate layer 12 may serve as the bottom of the biocompatible encapsulating housing 36 of the implantable sensor assembly 34. In this manner, as discussed above, the substrate layer 12 may be made of such materials as glass or liquid crystal polymer (LCP), or any other suitable material. The biocompatible encapsulating housing 36, including the substrate layer 12, may be made of any material approved to be implanted in the body (e.g., does not react with the body), having a low permeability to moisture or ions (e.g., near hermetic), having insulator capability (e.g., insulating dielectric), and may enable the implantable sensor assembly to be less than approximately 1 millimeter tall. As previously discussed, the insulating capability of the biocompatible encapsulating housing 36 may be described as an electrical resistivity (e.g., electrical resistance) of the substrate material. The electrical resistivity of the substrate material may indicate how strongly the material opposes a flow of electric current. In this manner, the biocompatible encapsulating housing 36 may resist the flow of electric current from the enclosed electronic components 28, and thus, protecting the surrounding environment. The electrical resistivity of the substrate material may be measured in Ohm-meters (Ωm), with a higher value corresponding to a higher electrical resistivity. For example, glass as a substrate material may have a relatively high resistivity value of approximately 10×10¹⁰ to 10×10¹⁴ Ωm, thus glass may have a high insulating capability. In a next step 52, the substrate layer 12 may be metallized with a metal layer 19 on the interior surface 15 (e.g., surface of substrate layer 12 that will be inside the implantable sensor assembly 34) of the substrate layer 12. In a next step 54, the metal layer 19 may be patterned to create a side feed-through connection 56. A cross section of the side feed-through connection 56 may be longer in length than a cross section of the metal layer 19 surrounding and filling the through via connections 16 of FIG. 1. It should be understood that patterning of the metal layer 19 may also be achieved through etching, deposition, and planarization in the process 50, as well as in the process 10 depicted in FIG. 1. In other embodiments, addition of the patterned metal layer 19 may be achieved additively, such as by screenprinting, inkjet printing, dispensejet printing, aerosol jet printing, or other suitable methods.

In a next depicted step 58, the cavity 24 may be formed through lamination of additional layers of the biocompatible material around most of the edges of the interior surface 15 of the substrate layer 12. This lamination step 22 may form sides 23 of the implantable sensor assembly 34 and thus, sides 23 of the cavity 24. The cavity 24 may be formed by the substrate layer and the sides 23, and may be the housing space for the electronic components 28 (e.g., signal processing and/or transmission electronics) of the implantable sensor assembly 34. Traditional lamination techniques may be used build the sides 23, and thus the cavity 24, to an expected depth. The side 23 through which the side feed-through connection 56 may extend, may be formed or laminated to the substrate layer 12 such that it abuts or surrounds the side feed-through connection 56. In this manner, a first end 57 of the side feed-through connection 56 may be within the cavity 24 and a second end 59, opposite the first end 57, of the side feed-through connection 56 may be outside of the cavity 24. The metal of the side feed-through connection 56 may enable the implantable sensor assembly 34 to be sealed, e.g., completely sealed to infiltration of external material or approximately completely sealed (permitting a negligible amount of infiltration within preset tolerances), while enabling connection between the enclosed electronic components 28 and outside electronics and/or other implantable sensor assemblies. That is, the biocompatible encapsulating housing 36 may form an enclosed shell interrupted only by any electrical connections extending continuously from a position within the cavity 24 to an exterior of the biocompatible encapsulating housing 36. In the illustrated embodiment, one side feed-through connection 56 is shown; however, the implantable sensor assembly 34 may have any quantity (e.g., 2, 3, 4, or more) of the side feed-through connections 56 through one or more of the sides 23.

In a next depicted step 60, the one or more electronic components 28 of the implantable sensor assembly 34 may be attached to the side feed-through connection 56 and/or the substrate layer 12 inside the cavity 24. As previously mentioned, the substrate layer 12 may serve as a circuit board for the attached electronic components 28 of the implantable sensor assembly 34. The one or more electronic components 28 may be positioned inside the cavity 24 and on top of the patterned metal, including the side feed-through connection 56. In this manner, the side feed-through connection 56 may enable connection between the electronic components 28 within the cavity 24 and other electronics and/or other implantable sensor assemblies outside of the implantable sensor assembly 34. In a next depicted step 62, the cavity 24 may be sealed by adding a cap layer 32. Like the substrate layer 12, the cap layer 32 may be made of glass or liquid crystal polymer (LCP), or any other material suitable for creating the biocompatible encapsulating housing 36. The cap layer 32 may be a flat layer, or may contain a recess that becomes an upper part of the cavity 24. The cap layer 32 may be added and the perimeter (e.g., substrate layer 12, sides 23, and cap layer 32) of the implantable sensor assembly 34 may be sealed using a localized perimeter sealing techniques. The localized perimeter sealing technique may be a technique that will not damage the encapsulated electronic components 28. For example, in some embodiments, the localized perimeter sealing technique may be a low temperature technique (e.g., laser seal) that does not use harsh solvents. In some embodiments, the localized perimeter sealing technique may concentrate the heat used in the sealing process in only the seal region, such that the heat is away from the encapsulated electronic components 28.

The resulting implantable sensor assembly 34 contains the substrate layer 12, laminated sides 23, and the cap layer 32, each made of glass or liquid crystal polymer (LCP), or any other material suitable for creating the biocompatible encapsulating housing 36. The implantable sensor assembly 34 contains the cavity 24 within the biocompatible encapsulating housing 36. Within the cavity 24, the implantable sensor assembly 34 contains the electronic components 28 attached to the substrate layer 12 and the patterned metal layer 19, including the side feed-through connection 56. The implantable sensor assembly 34 includes the side feed-through connection 56 formed through a side 23 and on the interior surface 15 of the substrate layer 12 of the implantable sensor assembly 34 that may enable a connection between the encapsulated electronic components 28 and other outside electronics and/or other implantable sensor assemblies. The implantable sensor assembly 34 may enable increased reliability of the sensor electronic components 28 by encapsulating and protecting the electronic components 28 and enabling connection to other outside electronics and/or other implantable sensor assemblies through the side feed-through connection 56, while maintaining a sealed enclosure. Further, the process 50 may enable increased modularity and improved manufacturability of the implantable sensor assembly 34.

FIG. 3 depicts a process flow (process 70) suitable for fabricating an embodiment of the implantable sensor assembly 34 incorporating a sensor module 72 (e.g. a sensor assembly on a substrate) built using traditional techniques. In this manner, sensor module 72 may be encapsulated within the biocompatible encapsulating housing 36 to form the implantable sensor assembly 34. At a first step, the sensor module 72 and the substrate layer 12 may be provided. The sensor module 72 may include the electronic components 28 of the implantable sensor assembly 34 assembled onto a substrate platform made of such materials as polyimide, ceramic, silicone, or any other substrate material traditionally used to create a substrate for an electronic sensor assembly. As previously discussed, the substrate layer 12 may serve as the bottom of the biocompatible encapsulating housing 36 of the implantable sensor assembly 34. As such, the substrate layer 12 may be made of such materials as glass or liquid crystal polymer (LCP), or any other suitable material. The biocompatible encapsulating housing 36, including the substrate layer 12, may be made of any material approved to be implanted in the body (e.g., does not react with the body), having a low permeability to moisture or ions (e.g., near hermetic), having insulator capability (e.g., insulating dielectric), and may enable the implantable sensor assembly to be less than approximately 1 millimeter tall.

In a next depicted step 74, the sensor module 72 may be place on the interior surface 15 of the substrate layer 12. The substrate layer 12 may be a flat surface, or may have a recess such that the module may be placed within the recess. The recess may form the cavity 24. The sensor module 72 may then be tacked in place on the interior surface 15 of the substrate layer 12 using an adhesive (e.g., ultraviolet (UV), PSA, epoxy). In some embodiments, the sensor module 72 may be tacked in place on the interior surface of the substrate layer 12 without an adhesive. For example, the sensor module 72 may be tacked in place using a localized heating method, such as a laser attachment method. In other embodiments, the sensor module 72 may be fitted to the cavity 24 in a manner such that no adhesive is needed. In such embodiments, sealing of the cavity 24 may hold the sensor module 72 in place and prevent the sensor module 72 from moving within the cavity 24.

At a next depicted step 76, the cap layer 32 may be added. Like the substrate layer 12, the cap layer 32 may be made of glass or liquid crystal polymer (LCP), or any other material suitable for creating the biocompatible encapsulating housing 36. The cap layer 32 may be a flat layer, or may contain a recess that becomes an upper part of the cavity 24. In some embodiments, if the substrate layer 12, the cap layer 32, or both are flat substrate surfaces, a lamination process may be performed such that additional layers of the substrate material are added around the edges of the interior surface 15 of the substrate layer 12 using a traditional lamination technique to form the sides 23 creating or increasing the depth of the cavity 24. Additionally or alternatively, the edges of the substrate layer 12 and/or the cap layer 32 having a recess may form the sides 23 of the implantable sensor assembly 34, and thus, the cavity 24. In a next depicted step 78, the substrate layer 12 and the cap layer 32 may be sealed using a localized perimeter sealing technique to form the biocompatible encapsulating housing 36 and the implantable sensor assembly 34. As previously discussed, the localized perimeter sealing technique may be a technique that will not damage the encapsulated electronic components 28. For example, in some embodiments, the localized perimeter sealing technique may be a low temperature technique (e.g., laser welding) that does not use harsh solvents. In some embodiments, the localized perimeter sealing technique may concentrate the heat used in the sealing process in only the seal region, such that the heat is away from the encapsulated electronic components 28.

The resulting implantable sensor assembly 34 contains the substrate layer 12, laminated sides 23, and the cap layer 32, each made of glass or liquid crystal polymer (LCP), or any other material suitable forming the biocompatible encapsulating housing 36. The cavity 24 within the biocompatible encapsulating housing 36 contains the sensor module 72, which may include sensor electronics on a substrate platform built using traditional techniques. Encapsulating the traditional sensor module 72 within the biocompatible encapsulating housing 36 of the implantable sensor assembly 34 may enable increased reliability of the sensor components by protecting the sensor module 72 from the environment outside of the implantable sensor assembly 34. Further, the process 70 may enable increased modularity and improved manufacturability of the implantable sensor assembly 34.

It should be understood that the processes 10, 50, and 70 shown in FIGS. 1-3 are examples of processes that may be used to fabricate the implantable sensor assembly 34. The processes 10, 50, and 70 may utilized alone or in combination to fabricate implantable sensor assemblies 34 having the through via connections 16, the side feed-through connections 56, the incorporated sensor module 72, or a combination thereof.

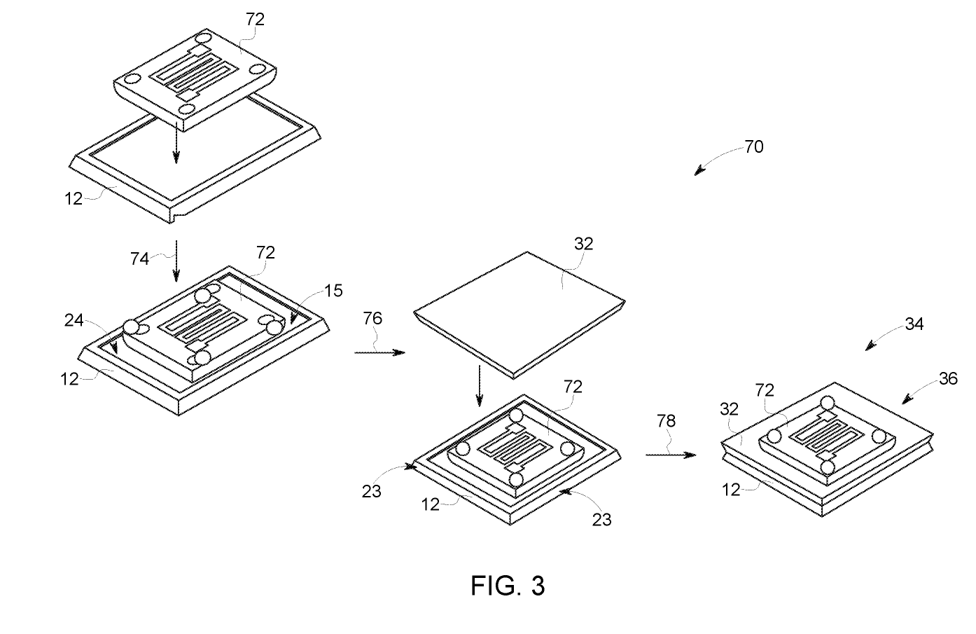

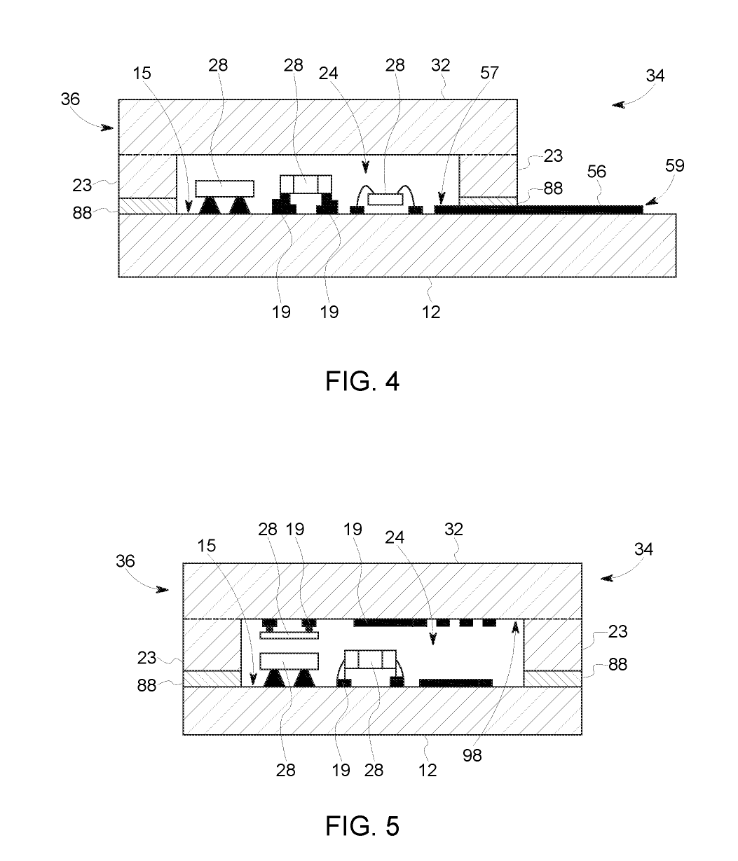

FIG. 4 is a cross section view of an embodiment of the implantable sensor assembly 34 having one or more of the side feed-through connections 56. The side feed-through connection 56 may be disposed on a top (e.g., interior) surface of the substrate layer 12 (e.g., platform layer). Additionally, the side feed-through connection 56 may be disposed such that the first end 57 of the side feed-through connection 56 is within the cavity 24 of the implantable sensor assembly 34 and the second end 59, opposite the first end 57, is outside of the cavity 24. In this manner, the side feed-through connection 56 may enable interconnection between the electrical components 28 within the implantable sensor assembly 24, as well as connection between the electrical components 28 within the implantable sensor assembly 34 and other outside electronics and/or other implantable sensor assemblies.

In the illustrated embodiment, the implantable sensor assembly 34 includes the substrate layer 12 and the cap layer 32 coupled together by seals 88, which form the biocompatible encapsulating housing 36. In some embodiments, the material of the seals 88 may be one or more of the material of the substrate layer 12, the side 23, or the cap layer in the case of a laser welding or other similar sealing technique. Further, insofar as, in certain embodiments, structures pass through channels/through vias sized to accommodate the structures and to the outside of the sensor assembly 34, such structures may also act to seal the channels. If electrical feedthroughs go under the seal 88, the components may be electrically insulating. Also either the cap 32 or the substrate 12 may have layers for attachment, connection and redistribution of components.

The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers only a portion of the interior surface 15 of the substrate layer 12, enabling the side feed-through connection 56 on the interior surface 15 of the substrate layer 12 to extend outside of the cavity 24 (e.g., end 59). In some embodiments, the cap layer 32 may cover the entire interior surface 15 of the substrate layer 12, thus the seals 88 may be disposed along the perimeter of the interior surface 15.

The electrical components 28 of the implantable sensor assembly 34 may be disposed on the interior surface 15 of the substrate layer 12 and within the cavity 24. The electrical components 28 may be enclosed within the biocompatible encapsulating housing 36 formed by the substrate layer 12, the cap layer 32, the sides 23, and the seals 88. The electrical components 28 may connect to each other, to outside electronics, and/or to other implantable sensor assemblies via the side feed-through connection 56. Thus, the implantable sensor assembly 34 may provide an environmental protection function to the electrical components 28, while enabling interconnection and connection to outside components, as well. In some embodiments, the substrate layer 12, the cap layer 32, and the sides 23 may be made of glass or liquid crystal polymer (LCP), or any other suitable material. Glass, as a substrate material, is low profile, biocompatible, has stable chemical properties, is compliant when thinned below 100 μm, and is capable of being sealed using a perimeter sealing technique. LCP, as a substrate material, is low profile, biocompatible, has stable chemical properties, is compliant, flexible, and is capable of being sealed using a perimeter sealing technique. These materials, or other materials suitable for forming the biocompatible encapsulating housing 36, may enable the implantable sensor assembly 34 to be thinner and lower profile than typical packaged sensing implant structures, thus enabling improved function and protection of the electrical components 28 of the implantable sensor assembly 34.

FIG. 5 is a cross section view of an embodiment of the implantable sensor assembly 34 that does not contain any through via connections 16 or any side feed-through connections 56. In the illustrated embodiment, the implantable sensor assembly 34 includes the substrate layer 12 and the cap layer 32 coupled together by seals 88, which form the biocompatible encapsulating housing 36. The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers all of the interior surface 15 of the substrate layer 12 and the seals 88 are disposed along the perimeter of the interior surface 15. This may allow the cavity 24 of the implantable sensor assembly 34 to be sealed from the surrounding environment.

In the illustrated embodiment, the electrical components 28 of the implantable sensor assembly 34 are attached to the interior surface 15 of the substrate layer 12, as well as on a bottom or interior surface 98 of the cap layer 32, within the cavity 24. In some embodiments, the electrical components 28 may be attached to the patterned metal layer 19 and/or the biocompatible encapsulating housing 36 on the interior surface 15 of the substrate layer 12, on the interior surface 98 of the cap layer 32, or a combination thereof. Thus, both the substrate layer 12 and the cap layer 32 may serve as circuit boards for the encapsulated electronic components 28 of the implantable sensor assembly 34, as well as environmental protection layers.

In some embodiments, the substrate layer 12, the cap layer 32, and the sides 23 of the implantable sensor assembly 34 may be made of glass or liquid crystal polymer (LCP), or any other suitable substrate material. Substrate materials such as glass may enable the biocompatible encapsulating housing 36 of the implantable sensor assembly 34 to be transparent. Optical transparency may enable the encapsulated electronic components 28 of embodiments of the implantable sensor assembly 34 without through via connections 16 and side feed-through connections 56, such as the illustrated embodiment, to connect to or communicate with outside probes, electronics, or other implantable sensor assemblies. These optical pathways created by the substrate material of the implantable sensor assembly 34 may enable improved environmental protection of the encapsulated electronic components 28 because the implantable sensor assembly 34 may be sealed with no feed-through connections, while enabling power and/or communication between the enclosed electrical components 28 and other outside components. Additionally or alternatively, in some embodiments, one or more of the substrate materials, such as glass, may be transparent to radio frequency (RF) waves. In such embodiments, the implantable sensor assembly 34 may contain an antenna attached to the substrate 12 or the cap layer 23 within the cavity 24. RF transparency of the substrate material may further enable power and/or communication between the encapsulated electronic components 28 of the implantable sensor assembly 34 and outside probes, electronics, and/or other implantable sensor assemblies. Additionally or alternatively, in some embodiments, one or more of the substrate materials may be transparent to, i.e., permit transmission of, electromagnetic energy. Electromagnetic transparency of the substrate material may further enable power and/or communication between the encapsulated electronic components 28 and outside probes, electronics, and/or other sensor assemblies.

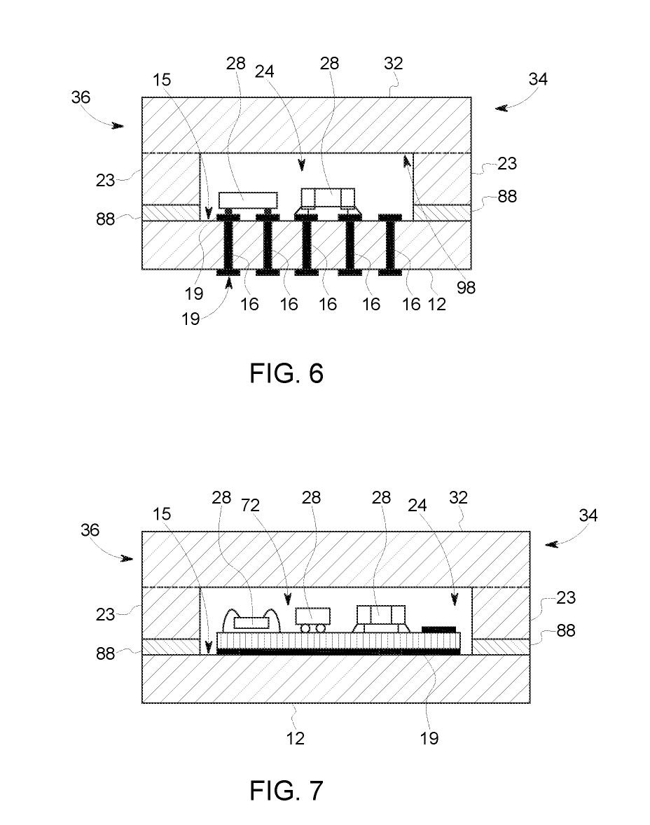

FIG. 6 is a cross section view of an embodiment of the implantable sensor assembly 34 showing five through via connections 16. In the illustrated embodiment, the implantable sensor assembly 34 includes the substrate layer 12 and the cap layer 32 coupled together by seals 88, which form the biocompatible encapsulating housing 36. The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers all of the interior surface 15 of the substrate layer 12 and the seals 88 are disposed along the perimeter of the interior surface 15.

In the illustrated embodiment, the implantable sensor assembly 34 includes multiple through via connections 16 disposed through the substrate layer 12 such that the through via connections 16 create pathways between the cavity 24 of the implantable sensor assembly 34 and the surrounding environment. The through via connections 16 may be filled with and surrounded at either end by a metal layer 19, or other conductive material. Metallization of and around the through via connections 16 may enable the implantable sensor assembly 34 to be sealed while enabling connection between the electrical components 28 of the implantable sensor assembly 34 and a coupled probe array, outside electronics, and/or other implantable sensor assemblies outside of the implantable sensor assembly 34.

The cavity 24 may house the electrical components 28 of the implantable sensor assembly 34. In the illustrated embodiment, the electrical components 28 are attached to the metal layer 19 and/or the interior surface 15 of the substrate layer 12 and positioned above the through via connections 16 within the cavity 24. Attachment of the electrical components 28 to the metal layer 19 may enable the electrical connection, discussed above, to outside electronics and/or sensor components, thus enabling the substrate layer to serve as a circuit board for the implantable sensor assembly 34. As previously discussed, in some embodiments, the electrical components 28 may be attached to the interior surface 98 of the cap layer 32, or to both the interior surface 15 of the substrate layer 12 and the interior surface 98 of the cap layer 32. In some embodiments, the encapsulated electrical components 28 of the implantable sensor assembly 34 within the cavity 24 may include surface mounted technology (SMT) electronics, application specific integrated circuit (ASIC) technology electronics, any other electronics suitable for sensing implant structures, or a combination thereof.

FIG. 7 is a cross section view of an embodiment of the implantable sensor assembly 34 incorporating the sensor module 72 (e.g. a sensor assembly on a substrate) built using traditional techniques. In the illustrated embodiment, the implantable sensor assembly 34 includes the substrate layer 12 and the cap layer 32 coupled together by seals 88, which form the biocompatible encapsulating housing 36. The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers all of the interior surface 15 of the substrate layer 12 and the seals 88 are disposed along the perimeter of the interior surface 15. This may allow the cavity 24 of the implantable sensor assembly 34 to be sealed from the surrounding environment.

In the illustrated embodiment, the sensor module 72 may be positioned within the cavity 24 on the metal layer 19 and/or the interior surface 15 of the substrate layer 12. The sensor module 72 may include the electronic components 28 of the implantable sensor assembly 34 assembled onto a substrate platform made of such materials as polyimide, ceramic, silicone, or any other substrate material traditionally used to create a substrate for an electronic sensor assembly. The biocompatible encapsulating housing 36 (e.g. the substrate layer 12, the cap layer 32, and sides 23) may enclose the sensor module 72 within the cavity 24 of the implantable sensor assembly 34. Encapsulating the traditional sensor module 72 within the biocompatible encapsulating housing 36 of the implantable sensor assembly 34 may enable increased reliability of the sensor components by protecting the sensor module 72 from the environment outside of the implantable sensor assembly. Further, embodiments of the implantable sensor assembly 34 including the traditional sensor module 72 encapsulated in the biocompatible encapsulating housing 36 may enable increased modularity and improved manufacturability of the implantable sensor assembly 34.

In the illustrated embodiment, the implantable sensor assembly 34 does not include any through via connections 16 or side feed through connections 56. In some embodiments, the implantable sensor assembly 34 incorporating the sensor module 72 built using traditional techniques may include one or more through via connections 16, one or more side feed through connections 56, or a combination thereof. In such embodiments, such as the illustrated embodiment, where no through via connections 16 or side feed-through connections 56 are present, properties of the substrate material may enable connection to outside electronics. In some embodiments, the substrate layer 12, the cap layer 32, and the sides 23 of the implantable sensor assembly 34 may be made of glass or liquid crystal polymer (LCP), or any other suitable substrate material. Substrate materials such as glass may enable optical pathways for the encapsulated electronic components 28 to connect to or communicate with outside probes and/or electronics. These optical pathways created by the substrate material of the implantable sensor assembly 34 may enable improved environmental protection of the encapsulated electronic components 28 because the implantable sensor assembly 34 may be sealed with no feed-through connections, while enabling communication between the enclosed electrical components 28 and outside electronics. Additionally or alternatively, in some embodiments, one or more of the substrate materials, such as glass, may be transparent to radio frequency (RF) waves, thus enabling communication between the encapsulated electronic components 28 of the implantable sensor assembly 34 and outside probes and/or electronics via an enclosed antenna, as previously discussed. These communication pathways (e.g., optical, RF) may be present in embodiments of the implantable sensor assembly 34 containing physical feed-through connections (e.g., through via connections 16, side feed-through connections 56) and in embodiments without physical feed-through connections. Accordingly, in certain embodiments, the enclosed electrical components 28 may include one or more of a transmitter, a receiver, a light emitter, or a photodetector, or other circuitry.

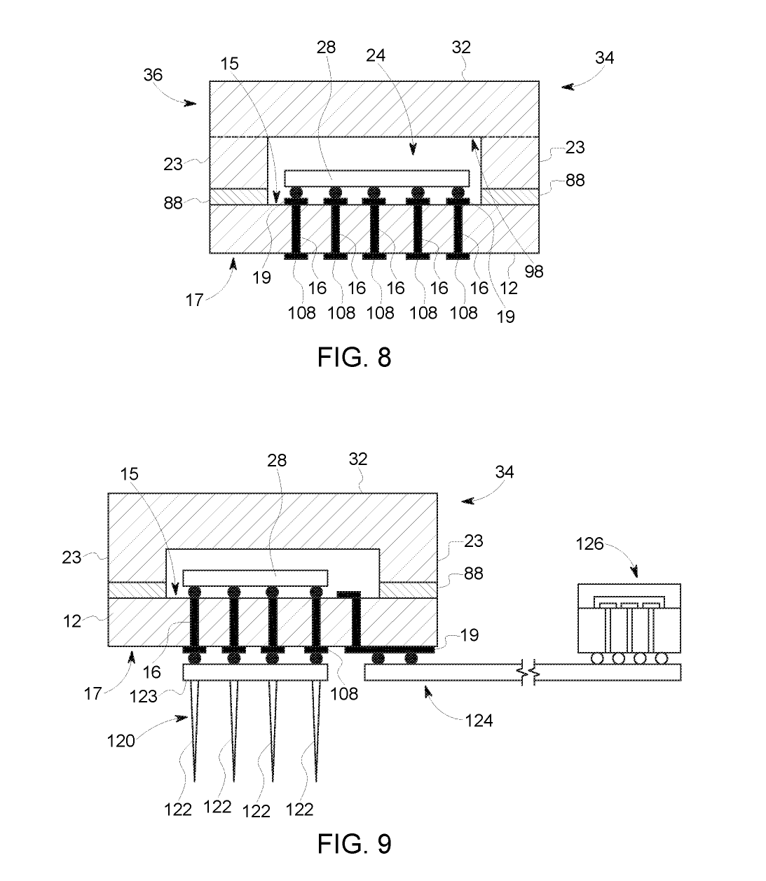

FIG. 8 is a cross section view of an embodiment of the implantable sensor assembly 34 showing five through via connections 16. In the illustrated embodiment, the implantable sensor assembly 34 includes the substrate layer 12 and the cap layer 32 coupled together by seals 88, which form the biocompatible encapsulating housing 36. The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers all of the interior surface 15 of the substrate layer 12 and the seals 88 are disposed along the perimeter of the interior surface 15.

In the illustrated embodiment, the implantable sensor assembly 34 includes multiple through via connections 16 disposed through the substrate layer 12 such that the through via connections 16 create pathways between the cavity 24 of the implantable sensor assembly 34 and the surrounding environment. As previously discussed, the through via connections 16 may be filled with and surrounded at either end by a metal layer 19, or other conductive material. Metallization of and around the through via connections 16 may enable the implantable sensor assembly 34 to be sealed while enabling connection between the electrical components 28 of the implantable sensor assembly 34 and a coupled probe array and/or outside electronics outside of the implantable sensor assembly 34. In the illustrated embodiment, the electrical components 28 are attached to the metal layer 19 and/or the interior surface 15 of the substrate layer 12 within the cavity 24 and positioned above the through via connections 16 within the cavity 24. In some embodiments, the encapsulated electrical components 28 of the implantable sensor assembly 34 within the cavity 24 may include surface mounted technology (SMT) electronics, application specific integrated circuit (ASIC) technology electronics, any other electronics suitable for sensing implant structures, or a combination thereof.

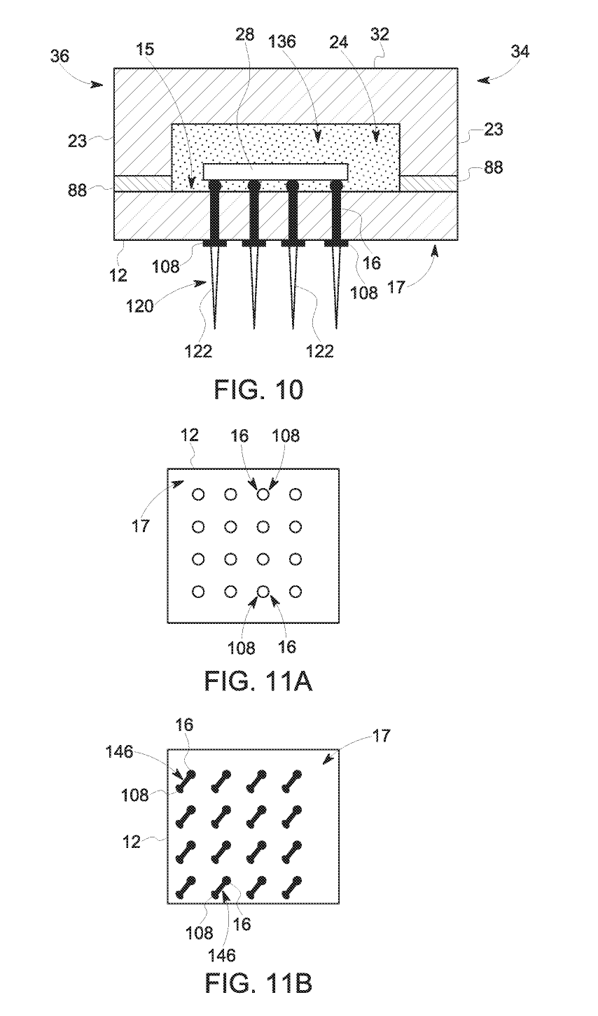

In some embodiments, the implantable sensor assembly 34 may include one or more pads 108 disposed on the exterior surface 17 of the substrate layer 12. In this manner, the pads 108 may be disposed on the exterior surface 17 of the substrate layer outside of the cavity 24 of the implantable sensor assembly 34. The pads 108 may serve as attachment sites for an attached probe array, as discussed in detail with reference to FIGS. 9 and 10. The pads 108 may be positioned such that they are aligned with the through via connections 16, as in the illustrated embodiment. The through via connections 16 may enable connection between the electronic components 28 of the implantable sensor assembly 34 enclosed in the cavity 24 and probes of a probe array that may be attached to the pads 108 aligned with, or adjacent to, the through via connections 16 on the exterior surface 17 of the substrate layer 12.

FIG. 9 is a cross section view of an embodiment of the implantable sensor assembly 34 including an attached probe array 120 and a connection 124 to outside electronic components 126. In the illustrated embodiment, the implantable sensor assembly 34 includes the substrate layer 12 and the cap layer 32 coupled together by seals 88, which form the biocompatible encapsulating housing 36. The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers all of the interior surface 15 of the substrate layer 12 and the seals 88 are disposed along the perimeter of the interior surface 15.

In the illustrated embodiment, the implantable sensor assembly 34 includes the through via connections 16 disposed through the substrate layer 12, creating electrical connection pathways between the cavity 24 and the environment surrounding the implantable sensor assembly. In some embodiments, the pads 108 may be disposed on the exterior surface 17 of the substrate layer 12 outside of the cavity 24 of the implantable sensor assembly 34. The pads 108 may be disposed aligned with or adjacent to the through via connections 16 on the exterior surface 17 of the substrate layer 12. The pads 108 may serve as attachment sites for an attached probe array 120. The probe array 12 may be attached to the pads 108 as a whole, using probe platform 123, or the probes 122 of the probe array may be attached individually. The probe array 120 may include any quantity (e.g., 1, 2, 3, 4, 5, or more) of probes 122 that may be used for measuring parameters of the surrounding environment of the implantable sensor assembly 34 when the implantable sensor assembly 34 is in use. The probes 122 of the probe array 120 may transmit to and/or receive information from the electronic components 28 of the implantable sensor assembly 34 via the connection pathways created by the through via connections 16.

In some embodiments, the through via connections 16 may enable a connection 124 to outside electronic components 126 outside of the implantable sensor assembly 34. The metal layer 19 that may be disposed within and around the through via connections 16 may provide a position for the connection 124 to various outside electronic components 126. The outside electronic components 126 may be used for recording and processing data collected, transmitted, and/or processed by the probes 122 of the probe array 120 and/or the electronic components 28 within the cavity 24 of the implantable sensor assembly 34. Thus, the implantable sensor assembly 34 may provide protection for the electronic components 28 within the cavity 24 while providing a platform for attachment of the probe array 120, as well as providing a method of connection between the encapsulated electronic components 28 and the outside electronic components 126.

FIG. 10 is a cross section view of an embodiment of the implantable sensor assembly 34 having the probes 122 connected to the exterior surface 17 of the substrate layer 12. In the illustrated embodiment, the implantable sensor assembly 34 includes the substrate layer 12 and the cap layer 32 coupled together by seals 88, which form the biocompatible encapsulating housing 36. The cap layer 32 includes a recess, which forms the sides 23 of the implantable sensor assembly and the cavity 24 with the interior surface 15 of the substrate layer 12. In some embodiments, the cap layer 32 may be have a flat structure. In such embodiments, the sides 23 of the implantable sensor assembly 34 may be formed by one or more layers of the substrate material laminated onto the interior surface 15 of the substrate layer 12, as previously discussed. In the illustrated embodiment, the cap layer 32 covers all of the interior surface 15 of the substrate layer 12 and the seals 88 are disposed along the perimeter of the interior surface 15. The substrate layer 12, the cap layer 32, and the sides 23 may form the biocompatible encapsulating housing 36 of the implantable sensor assembly 34. The cavity 24 within the biocompatible encapsulating housing 36 may house the electronic components 28 of the implantable sensor assembly 34. In some embodiments, the implantable sensor assembly 34 may include a phase-change material 136 within the cavity 24. The phase-change material my surround the electronic components 28 and fill the cavity 24. The phase-change material 136 may enable effective heat dissipation within the implantable sensor assembly if the electronic components 28 within the cavity 24 produce excess heat, thus protecting the surrounding environment and the electronic components 28. In some embodiments, the cavity 24 may contain dry air, inert gas, a vacuum, or dielectric liquids to ensure an environment that avoids any long term damage to the electronics.

In some embodiments, the probe array 120 may be attached to the exterior surface 17 of the substrate layer 12. The probes 122 may be positioned aligned with or adjacent to the through via connections 16 such that the through via connections 16 may enable connection and communication between the probes 122 and the electronic components 28 of the implantable sensor assembly 34 enclosed within the cavity 24. As previously discussed, in some embodiments, the probe array 120 may be attached to the pads 108 as a whole (e.g., as an assembly of multiple probes 122) using probe platform 123. In some embodiments, the probes 122 of the probe array 120 may be attached individually to the pads 108. Alternatively, the probes 122 may be positioned on the pads 108 on the exterior surface 17 of the substrate layer 12 by processes other than by attachment of the assembled probes 122 or the assembled probe array 120. The probes 122 may be grown onto the pads 108, printed onto the pads 108 (e.g., via a 3D printing process), deposited onto the pads 108 (e.g., sputtering, glance angle deposition), wire bonded onto the pads 108, or a combination thereof. The various techniques that may be used to assembly the probes 122 and/or the probe array 120 on the pads 108 on the exterior surface 17 of the substrate layer 12 may enable increased modularity and improved manufacturability of the implantable sensor assembly 34.

FIGS. 11A and 11B are bottom views of embodiments of the implantable sensor assembly 34 showing possible arrangements between the through via connections 16 and the pads 108 on the exterior surface 17 of the substrate layer 12. In the illustrated embodiments, the implantable sensor assembly 34 includes the through via connections 16 disposed through the substrate layer 12, creating electrical connection pathways between the cavity 24 and the environment surrounding the implantable sensor assembly 34. In some embodiments, the pads 108 may be disposed on the exterior surface 17 of the substrate layer 12 outside of the cavity 24 of the implantable sensor assembly 34. The pads 108 may be disposed aligned with, as illustrated in FIG. 11A, or adjacent to, as illustrated in FIG. 11B, the through via connections 16 on the exterior surface 17 of the substrate layer 12. The pads 108 may serve as attachment, growth, or deposition sites for the probes 122 and/or the probe array 120.

In some embodiments, as illustrated in FIG. 11A, the pads 108 may be directly aligned with the through via connections 16 such that the pads 108 cover the through via connections 16 on the exterior surface 17 of the substrate layer 12 of the implantable sensor assembly 34. This configuration may enable a direct connection between the attached probes 122 and/or the probe array 120 and the electronic components 28 of the implantable sensor assembly 34 within the cavity 24. In some embodiments, as illustrated in FIG. 11B, the pads 108 may be positioned adjacent to the through via connections 16 such that the pads 108 do not cover the through via connections 16 on the exterior surface 17 of the substrate layer 12. In some embodiments, the topography of the metalized through via connection 16 may not be desirable for attachment, growth, or deposition for the probes 122. In such embodiments, the through via connections 16 may be coupled to the pads 108 via couplings 146. This configuration may enable connection between the probes 122 and the electronic components 28 of the implantable sensor assembly 34 via the through via connections 16 and the coupling 146. Further, this configuration may enable adjustment of the connection or coupling between the electronic components 28 within the cavity 24 of the implantable sensor assembly and the attached probes 122 and/or probe array 120.

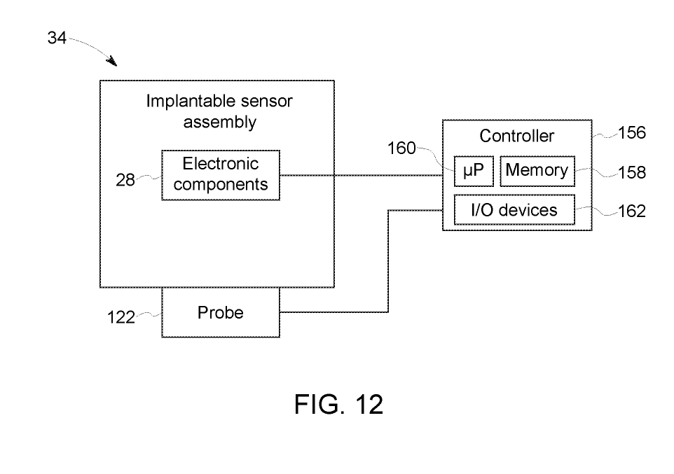

FIG. 12 is a schematic diagram of an embodiment of a control system that may be employed within the implantable sensor assembly 34. A controller 156 (e.g., electronic controller) of the implantable sensor assembly 34 may be configured to receive input from the probes 122 (e.g., sensors) and/or the electronic components 34 within the cavity 24. In some embodiments, the controller 156 may be configured to be positioned remote from the implantable sensor assembly 34. In other embodiments, the controller 156 may be configured to be positioned within the implantable sensor assembly 34. The controller 156 may include a memory 158, a processor 160, and input/output (I/O) devices 162. In some embodiments, the memory 158 may include one or more tangible, non-transitory, computer-readable media that store instructions executable by the processor 160 and/or data to be processed by the processor 160. For example, the memory 158 may include random access memory (RAM), read only memory (ROM), rewritable non-volatile memory such as flash memory, hard drives, optical discs, and/or the like. Additionally, the processor 68 may include one or more general purpose microprocessors, one or more application specific processors (ASICs), one or more field programmable logic arrays (FPGAs), or any combination thereof. The I/O devices 162 may facilitate communication between the controller 156 and a user (e.g., operator). For example, the I/O devices 70 may include a button, a keyboard, a mouse, a trackpad, and/or the like to enable user interaction with the controller 156 and the implantable sensor assembly 34. Additionally, the I/O devices 162 may include an electronic display to facilitate providing a visual representation of information, for example, via a graphical user interface (GUI), an application interface, text, a still image, and/or video content.

The controller 156 may be configured to receive input signals, via the processor 160, from the electronic components 28 and/or the probes 122 attached to the implantable sensor assembly 34 indicative of measured parameters of the environment surrounding the implantable sensor assembly 34. For example, the probes 122 may transmit measured parameters, such as neural signals, to the electronic components 28 where the signals may be processed and transmitted to the controller 156 and/or the outside electronic components 126. Additionally, the controller 156 may output signals to the electronic components 28 and/or the probes 122 instructing the implantable sensor assembly to take measurements of particular parameters or to transmit particular signals to the environment surrounding the implantable sensor assembly 34. In some embodiments, the received input signals and/or any control signals sent by the controller 156 may be saved in the memory 158. In some embodiments, indications of the input signals and/or the control signals may be displayed to an operator via a display of the I/O devices 162. In some embodiments, the I/O devices 162 may be used by an operator to provide instructions to the controller 156 to control parameters measured by the implantable sensor assembly 34, when such parameters are measured, and/or transmission of signals to the environment surrounding the implantable sensor assembly 34. Such control of the implantable sensor assembly 34 may enable an increase in the effectiveness of the implantable sensor assembly 34 in medical treatment and diagnosis.

Technical effects of the disclosed embodiments include providing an implantable sensor assembly having a cavity for enclosing electronic components of the implantable sensor assembly and methods for fabricating such implantable sensor assemblies. Thus, the implantable sensor assembly may have dual functionality as a protection layer to protect the enclosed electronic components from the environment surrounding the implantable sensor assembly and as a circuit board for the enclosed electronic components, thus enabling increased reliability of the implantable sensor assembly. The substrate material of the implantable sensor assembly (e.g., glass, LCP) may enable the implantable sensor assembly to be biocompatible, low profile, and flexible, thus improving use of the implantable sensor assembly for chronic implantation. Further, the disclosed methods of fabrication of the implantable sensor assembly may enable increased modularity and improved manufacturability, thus enabling a reduction in cost of the implantable sensor assembly.

Additionally, the implantable sensor assembly may include one or more through via connections through the substrate layer of the implantable sensor assembly, one or more side feed-through connections, or a combination thereof. These pathways may enable a connection between the enclosed electronic components and the attached probes, outside electronic components, and/or other implantable sensor assemblies, while enabling the cavity of the implantable sensor assembly to remain sealed from the surrounding environment. Additionally or alternatively, the substrate material of the implantable sensor assembly may be optically transparent and/or transparent to radio frequency (RF) waves, thus enabling connection to the probes, outside electronic components, and/or other implantable sensor assemblies in embodiments that do not include the through via connections or the side feed-through connections. These connection pathways (e.g., physical feed-through, optical, RF) may enable connections between the enclosed electronic components and the probes, outside electronics, and/or other implantable sensor assemblies without the use of bulky wire bundles, as the probes may be attached, grown, or deposited directly onto or adjacent to the through via connection sites. Further, in some embodiments, the implantable sensor assembly may include a phase-change material within the cavity to absorb any excess heat from the electronics, thus protecting the surrounding environment and the electronic components. The cavity could, in various embodiments, contain dry air, inert gas, a vacuum, or dielectric liquids to ensure an environment that avoids any long term damage to the electronics.

This written description uses examples to disclose the concepts discussed herein, including the best mode, and also sufficient disclosure to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A system, comprising:
an implantable sensor assembly comprising:
a housing comprising:
a substrate layer comprising an interior surface and an exterior surface; and
a cap layer, wherein the substrate layer and the cap layer are coupled to form an enclosed cavity that at least partially encloses the interior surface of the substrate layer within the cavity and wherein both the substrate layer and the cap layer are formed from an electrically insulating material;
one or more electronic components disposed within the cavity of the housing; and
one or more probes disposed on the exterior surface of the substrate layer and electrically coupled to the one or more electronic components by one or more electrical connections extending through the housing.

2. The system of claim 1, wherein the one or more electronic components are disposed directly on the interior surface of the substrate layer.

3. The system of claim 1, wherein the at least some of the one or more electronic components are disposed directly on an interior surface of the cap layer.

4. The system of claim 1, comprising a side-feed through connection disposed on the interior surface of the substrate layer such that a first end of the side feed-through connection is disposed within the cavity and a second end of the side feed-through connection is disposed outside of the cavity, wherein the second end is opposite the first end, and wherein the side-feed through connection is configured to electrically connect the one or more electronic components within the cavity to the one or more probes or other electrical components outside of the cavity.

5. The system of claim 1, wherein the one or more electrical connections comprise a plurality of through via connections disposed through the substrate layer such that each of the through via connections of the plurality of through via connections extends from the interior surface of the substrate layer to the exterior surface of the substrate layer.

6. The system of claim 5, wherein each of the through via connections of the plurality of through via connections is metallized such that a metal layer extends through and fills passageways to form each of the through via connections.

7. The system of claim 5, wherein the implantable sensor assembly comprises one or more pads disposed on the bottom surface of the substrate layer such that the each respective pad of the one or more pads is disposed between the exterior surface of the substrate layer and a respective probe of the one or more probes.

8. The system of claim 7, wherein the one or more pads are each aligned with a respective through via connection of the plurality of through via connections.

9. The system of claim 1, wherein the substrate layer and the cap layer each comprise one of glass or liquid crystal polymer (LCP), and wherein the implantable sensor assembly is less than 1 millimeter across at least one dimension.

10. The system of claim 1, wherein the housing forms an enclosure interrupted only by the electrical connections, and wherein the housing is biocompatible.

11. A system, comprising:
an implantable sensor assembly comprising:
a substrate layer;
a cap layer comprising a recess on one side, wherein cap layer and substrate layer are formed from electrically insulating materials;
a cavity formed between the substrate layer and the cap layer, wherein the recess of the cap layer forms part of the cavity;
a seal formed between the substrate layer and the cap layer, wherein the seal is configured to seal the cavity;
one or more electronic components disposed on a substrate platform, wherein the substrate platform is disposed on an interior surface of the substrate layer within the cavity; and
one or more probes disposed on an exterior surface of the substrate layer such that the probes are outside of the cavity.

12. The system of claim 11, wherein the implantable sensor assembly comprises a plurality of through via connections disposed through the substrate layer such that each of the through via connections of the plurality of through via connections extends from the interior surface of the substrate layer to the exterior surface of the substrate layer, wherein each of the through via connections of the plurality of through via connections is metallized such that a metal layer extends through and fills each of the through via connections.

13. The system of claim 11, wherein the implantable sensor assembly comprises a phase-change material disposed within the cavity.

14. The system of claim 11, wherein the implantable sensor assembly comprises one or more pads disposed on the exterior surface of the substrate layer between the exterior surface of the substrate layer the one or more probes.

15. The system of claim 11, wherein the substrate layer and the cap layer each comprise one of glass or liquid crystal polymer (LCP), and wherein the implantable sensor assembly is less than 1 millimeter across at least one dimension.

16. The system of claim 15, wherein the one or more electronic components are configured to communicate with separate electronic components outside of the implantable sensor assembly via optical waves or radio frequency (RF) waves.

17. A method for fabricating an implantable sensor assembly, comprising:
providing a substrate layer, wherein the substrate layer comprises one of glass, fused silica, quartz, sapphire, or liquid crystal polymer (LCP);
attaching electronic components directly to a first surface of the substrate layer;
sealing a cap layer over the first surface of the substrate layer to create a cavity between the substrate layer and the cap layer, wherein the sealing comprises a low temperature perimeter sealing technique, and wherein the cap layer comprises one of glass, fused silica, quartz, sapphire, or liquid crystal polymer (LCP); and
coupling one or more probes to a second surface of the substrate layer opposing the first surface and such that the one or more probes are disposed outside of the cavity.

18. The method of claim 17, comprising:
providing one or more passageways through the substrate layer and configured to accommodate one or more through via connections extending from the first surface of the substrate layer to the second surface of the substrate layer;
metallizing the top surface and the bottom surface of the substrate layer with a metal layer; and
patterning the metal layer such that the metal layer fills and surrounds the one or more to form the one or more through via connections.

19. The method of claim 17, comprising:
metallizing the top surface of the substrate layer with a metal layer;
patterning the metal layer creating a side feed-through connection;
building one or more sides of the cavity via lamination of one or more layers of the substrate material such that a first end of the side feed through connection is within the cavity and a second end of the side feed-through connection is outside of the cavity, wherein the second end is opposite the first end, wherein the cap layer is sealed onto the sides of the cavity.

20. The method of claim 17, wherein coupling the one or more probes to the bottom surface of the substrate layer comprises growing the probes, 3D printing the probes, depositing the probes via sputtering, wire bonding the probes to the substrate layer, or a combination thereof.
